(19) United States
(12) Patent Application Publication
(10) Pub. No.: US 2022/0061874 A1
(43) Pub. Date: Mar. 3, 2022

(54) Suction Tube with Blade

(71) Applicant: Yamaga CL LLC, Kumamoto (JP)

(72) Inventor: Kengoh TANAKA, Kumamoto (JP)

(21) Appl. No.: 17/281,233

(22) PCT Filed: Dec. 3, 2019
(86) PCT No.: PCT/JP2019/047178

§ 371 Date: Nov. 2, 2021

(30) Foreign Application Priority Data
Dec. 4, 2018 (JP) 2018-227700

(51) Int. Cl. A61B 17/32; A61M 1/00

(52) U.S. Cl. CPC A61B 17/32002; A61B 2217/005; A61M 1/60

(57) ABSTRACT

[Problem] To provide a medical device with which resection of tissue is possible in addition to collection of liquid. [Solution] A medical suction tube 1 includes a tubular body 3 and a connecting part 7 that connects the body 3 and a suction device 5. A blade 9 for resecting tissue is provided inside the distal-end area of the body 3.

### TECHNICAL FIELD

The present invention relates to a medical suction tube. More specifically, the present invention relates to a suction tube with a blade inside the tube.

### BACKGROUND ART

JPH08-336595A discloses a suction tube with a blade.

### CITATION LIST

### Patent Literature

PTL 1: JPH08-336595A

### SUMMARY OF THE INVENTION

### Technical Problem

Suction tubes are generally used only to absorb the target liquid during the operation. On the other hand, especially in the case of an operation in a narrow affected area where it is difficult to open and close scissors, it is sometimes desired to resect the tissue and object while absorbing body fluids with the suction tube. In such cases, replacing the medical device is cumbersome. For this reason, there has been a demand for medical devices that allow not only the collection of body fluids but also the resection of tissue.

### Solution to Problem

The present invention is basically based on the finding that when a blade facing toward the center is provided inside a thin medical suction tube, an object to be resected is guided into the suction tube, and the suction tube is rotated, whereby the object to be resected can be easily resected, and the resected object can be easily collected.

The first invention disclosed in this specification relates to a medical suction tube 1 (suction tube 1).

The suction tube 1 is a medical suction tube 1 having a tubular body part 3 and a connecting part 7 that connects the body part 3 to a suction device 5.

A blade 9 for resecting tissue is provided inside the tip region of the body part 3.

A preferred example of the suction tube 1 is as follows.

The blade 9 is a hollow blade that is present inside the body part at an angle with respect to a central axis 11 of the body part, and the hollow blade has an edge in contact with the inside of the body part.

The tip region of the body part has a shape inclined with respect to the central axis of the body part along the blade.

A preferred example of the suction tube 1 is as follows.

The blade has one or more blade parts with edges in contact with the inside of the body part.

A preferred example of the suction tube 1 is as follows.

The blade has a plurality of blade parts with edges in contact with the inside of the body part, and the plurality of blade parts are inclined with respect to the central axis of the body part.

A preferred example of the suction tube 1 is as follows.

A rotation mechanism for rotating the body part is contained.

A preferred example of the use of the suction tube 1 is a tissue collection device described below.

The tissue collection device comprises the suction tube 1, and a solution tank that is connected to the body part and contains a sucked object.

### Advantageous Effects of the Invention

Since the suction tube of the present invention has a blade facing toward the center inside the thin medical suction tube, an object to be resected is guided into the suction tube, and the suction tube is rotated, whereby the object to be resected can be easily resected, and the resected object can be easily collected.

### BRIEF DESCRIPTION OF THE DRAWINGS

FIG. 1 is a conceptual diagram showing an example of the use of a suction tube.

FIG. 2 is a conceptual diagram showing an example of the tip part of a suction tube. FIG. 2A shows a cross-sectional view of the tip part of the suction tube, and FIG. 2B shows an X-X cross-sectional view of FIG. 2A.

FIG. 3 is a conceptual diagram showing an example of a cutting blade part extending at an angle from the direction perpendicular to a central axis of the body part. FIG. 3A shows an example of a cutting blade part extending at an angle upward from the direction perpendicular to the central axis of the body part, and FIG. 3B shows an example of a cutting blade part extending at an angle downward from the direction perpendicular to the central axis of the body part.

FIG. 4 is a conceptual diagram showing an example of a suction tube having a blade that is a hollow blade present inside the body part at an angle with respect to the central axis of the body part, and having an edge in contact with the inside of the body part.

FIG. 5 is a conceptual diagram showing an example of a suction tube having a plurality of blade parts. FIG. 5A is a conceptual diagram of the suction tube being divided in half. FIG. 5B is a view of the suction tube of FIG. 5A viewed from above.

FIG. 6 is a conceptual diagram showing an example of a plurality of (three) inclined cutting blade parts extending at an angle from the direction perpendicular to the central axis of the body part. FIG. 6A shows a conceptual diagram of the suction tube viewed from above, FIG. 6B shows an X-X cross-sectional view of FIG. 6A, and FIG. 6C shows a Y-Y cross-sectional view of FIG. 6A.

FIG. 7 is a conceptual diagram showing an example of a suction tube comprising a rotation mechanism for rotating the body part.

### DESCRIPTION OF THE EMBODIMENTS

Modes for carrying out the present invention will be described below with reference to the drawings. The present invention is not limited to the modes described below, and includes those appropriately modified by a person skilled in the art within a scope obvious from the following modes.

The first invention disclosed in this specification relates to a medical suction tube 1 (suction tube 1). There are various suction tubes depending on the medical field (e.g., for otolaryngology, dentistry, ophthalmology, lacrimal surgery, urology, surgery, endoscopic surgery, and laparoscopic surgery) or the intended use (e.g., for coagulation tissue collection, necrotic tissue collection, secretion collection, mucus collection, blood collection, tear collection, and saliva collection).

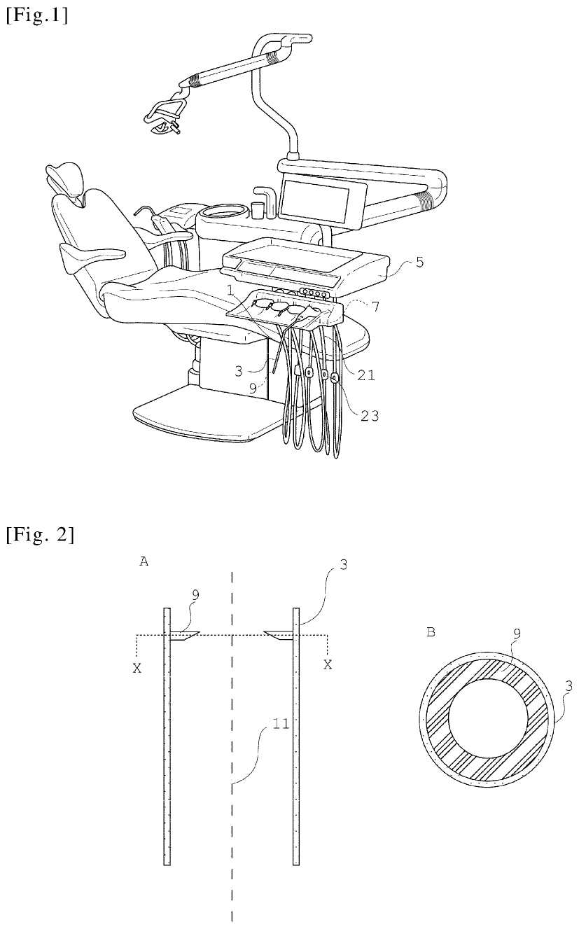

FIG. 1 is a conceptual diagram showing an example of the use of a suction tube. FIG. 2 is a conceptual diagram showing an example of the tip part of a suction tube. FIG. 2A shows a cross-sectional view of the tip part of the suction tube, and FIG. 2B shows an X-X cross-sectional view of FIG. 2A. The suction tube 1 is a medical suction tube 1 having a tubular body part 3 and a connecting part 7 that connects the body part 3 to a suction device 5. Suction tubes have body parts in various shapes, such as a tubular linear shape, a cylindrical shape with a part whose diameter decreases toward the tip, and a tubular shape with a bent part in the middle. The diameter φ of the tubular body part 3 is, for example, 0.5 mm or more and 1 cm or less, and may be 0.5 mm or more and 5 mm or less, or 1 mm or more and 3 mm or less. Since the present invention is preferably used in a minute region where it is difficult to open and close medical scissors, the diameter φ may be 0.5 mm or more and 2 mm or less. As shown in FIG. 2, a blade 9 for resecting tissue is provided inside the tip region of the body part 3. All or part of the edge (peak) of the blade 9 is preferably connected to the inner wall of the body part 3 of the suction tube. It is preferable that the part (cutting blade part) that cuts the target site, such as tissue, faces toward a central axis of the suction tube inside the suction tube. The cutting blade part may extend in the direction perpendicular to a central axis of the body part 3, or may extend at an angle upward or downward.

The pressure of the suction tube becomes negative upon suction from the suction device. Therefore, as shown in FIG. 2A, the blade 9 preferably has a taper (inclination) such that the width of the blade increases toward the tip (the upper part of the drawing) (the cutting blade part extends at an angle upward with respect to the central axis of the body part 3). The suction force can be increased by the possession of such a blade. On the other hand, as shown in FIG. 2B, when the blade has a hollow shape (a shape in which there is no blade around the central axis), there is a portion above the blade 9 where the suction from the suction device is weakened. If there is microtissue that is not desired to be sucked, the microtissue can be positioned above the blade 9, thereby protecting the tissue while sucking body fluids, which is preferable. From such a viewpoint, the blade length (blade width: distance from the inner wall of the suction tube to the blade edge) is, when the inner diameter of the suction tube is regarded as φ, is preferably 1/20 or more and 1/3 or less of φ, and may be 1/10 or more and 1/4 or less, or 1/10 or more and 1/5 or less. Since the suction tube is very small, the thickness of the blade may be adjusted accordingly. For example, the thickness of the blade may be 10 μm or more and 1 mm or less, 50 μm or more and 0.5 mm or less, or 0.1 mm or more and 0.5 mm or less. The blade 9 may be present at the tip of the suction tube, or as shown in FIG. 2A, below the tip to secure the top of the blade and avoid blade loss. The blade of FIG. 2B has a hollow circular shape. Alternatively, the blade may be chord-shaped, or may not be a perfect circle but may be in a state of being partially present, such as a distorted circle or a tooth mark.

In the example shown in FIG. 1, the suction tube 1 is connected to the suction device 5 via a hose 21. The hose 21 is provided in the middle with a hole 23 that allows the suction state to continue when closed with a practitioner finger, and weakens the suction force when opened. The hole 23 may be present in a hose portion, such as the hose 21, that connects the suction tube to the suction device. Alternatively, this hole may be present in the connecting part 7 of the suction tube or in the body part on the connecting part 7 side. Since the suction tube has the connecting part 7, it is preferable that the suction tube can be removed from the hose etc., it is preferable that the suction tube 1 can be washed, and it is preferable that the suction tube can be replaced.

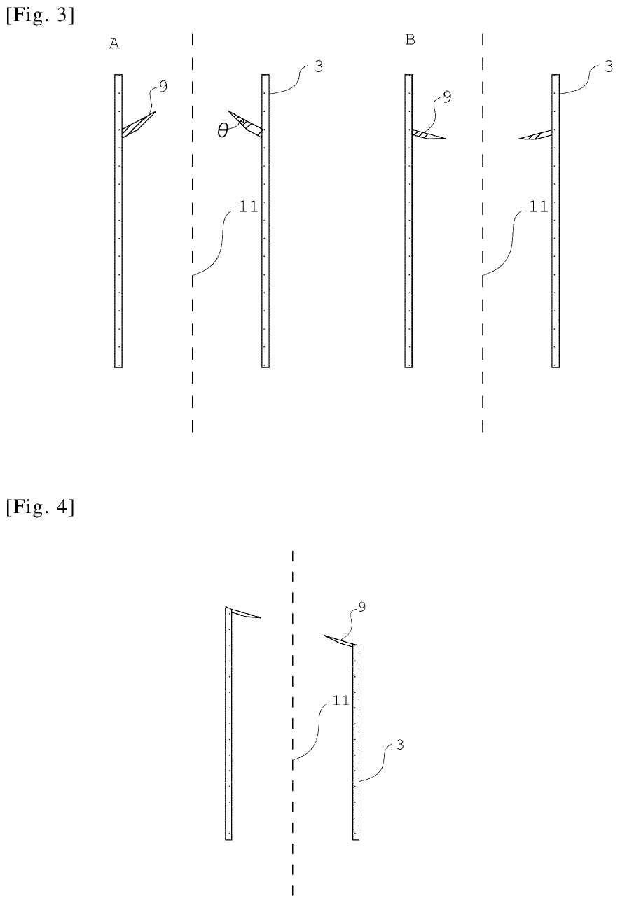

FIG. 3 is a conceptual diagram showing an example of a cutting blade part extending at an angle from the direction perpendicular to the central axis of the body part. FIG. 3A shows an example of a cutting blade part extending at an angle upward from the direction perpendicular to the central axis of the body part, and FIG. 3B shows an example of a cutting blade part extending at an angle downward from the direction perpendicular to the central axis of the body part. In FIG. 2, the blade 9 extends substantially perpendicular from the inner wall of the body part 3, while in FIG. 3, the blade is inclined. In FIG. 3A, θ indicates the taper angle of the cutting blade part, and θ is, for example, 1 degree or more and 45 degrees or less, and may be 5 degrees or more and 30 degrees or less, or 5 degrees or more and 20 degrees or less.

FIG. 4 is a conceptual diagram showing an example of a suction tube having a blade that is a hollow blade present inside the body part at an angle with respect to the central axis of the body part, and having an edge in contact with the inside of the body part. In this example, the tip of the suction tube is cut diagonally like an injection needle, and a blade 9 is present along the cut tip. Alternatively, the tip part may be tubular. The blade 9 is a hollow blade that is present inside the body part at an angle with respect to the central axis 11 of the body part, and has an edge in contact with the inside of the body part, whereby a small object can be easily resected. When the tip region of the body part has a shape inclined with respect to the central axis of the body part along the blade, the suction tube itself can be used like an injection needle, and the suction tube body part can be used to resect the object or to continuously suck body fluids, such as blood, while being inserted into the tissue. The inclination may have any angle. Assuming that an angle of 0 degrees is perpendicular to the central axis 11, the inclination is, for example, 1 degree or more and 70 degrees or less, and may be 5 degrees or more and 45 degrees or less, 10 degrees or more and 30 degrees or less, or 10 degrees or more and 50 degrees or less.

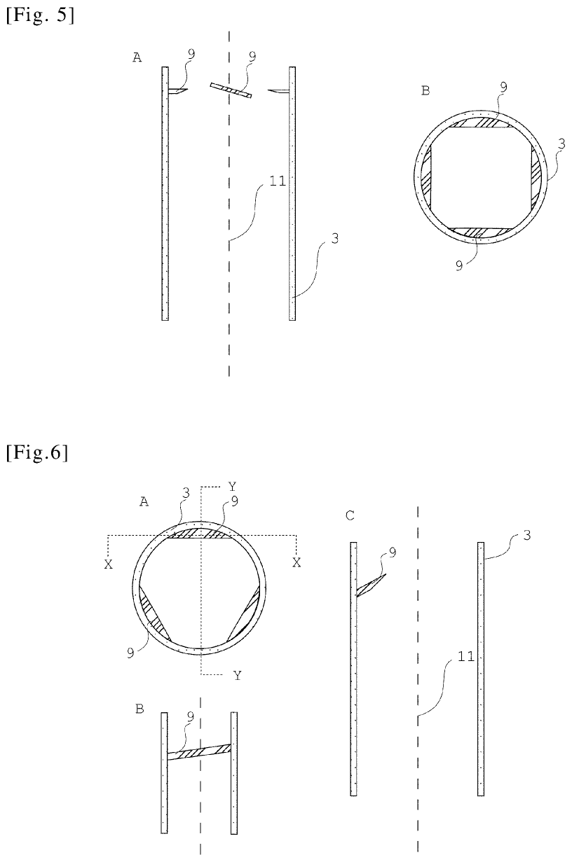

The edge of the blade is preferably in contact with the inside of the body part. The number of blades may be one, or there may be a plurality of blade parts. FIG. 5 is a conceptual diagram showing an example of a suction tube having a plurality of blade parts. In the example shown in FIG. 5, the blade has a plurality of blade parts with edges in contact with the inside of the body part, and the plurality of blade parts are inclined with respect to the central axis of the body part. Having such a shape makes it possible to easily resect the target tissue and the like. FIG. 5A is a conceptual diagram of the suction tube being divided in half. FIG. 5B is a view of the suction tube of FIG. 5A viewed from above. In the example of FIG. 5, four blade parts are provided every 90 degrees. The blade edges are present in parts facing toward the central axis, and the object, such as tissue, inserted into the suction tube can be resected. When the suction tube has a plurality of blades 9, the plurality of blades 9 may be inclined in the same direction (when the body part 3 is developed, the orientations of the blades 9 are approximately parallel to each other, like /,/,/,/), or the adjacent blades 9 may be inclined in opposite directions (when the body part 3 is developed, the orientations of the blades 9 are like /,\,/,\).

FIG. 6 is a conceptual diagram showing an example of a plurality of (three) inclined cutting blade parts extending at an angle from the direction perpendicular to the central axis of the body part. FIG. 6A shows a conceptual diagram of the suction tube viewed from above, FIG. 6B shows an X-X cross-sectional view of FIG. 6A, and FIG. 6C shows a Y-Y cross-sectional view of FIG. 6A. When the body part of the suction tube is rotated, the target tissue can be easily resected by the cutting blade parts of the blades.

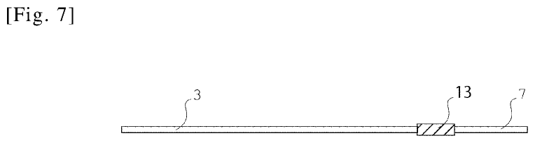

FIG. 7 is a conceptual diagram showing an example of a suction tube comprising a rotation mechanism for rotating the body part. The rotation mechanism is an optional element; however, the presence of the rotation mechanism has an advantageous effect as explained below. For example, the rotation mechanism 13 is hollow, and does not prevent the suction of liquid or objects by the suction tube. This suction tube may have a hole to relieve pressure. In an example of the rotation mechanism 13, when the rotation mechanism 13 is rotated with a finger or a hand using a rotation drive mechanism, such as a ball bearing, the body part 3 can be driven to rotate relatively with respect to the connecting part 7, along with the rotation of the rotation mechanism. When the rotation mechanism 13 is moved with a finger, the blade rotates in accordance with the rotation of the body part of the suction tube; thus, the target tissue can be easily resected during the operation. Further, even if the suction of the suction tube is reduced during the operation, the rotation mechanism 13 can be rotated to thereby give vibration to the body part and prevent clogging, which also contributes to the efficiency of the operation. In addition, a small motor or actuator may be used to drive the body part 3 to rotate with respect to the connecting part 7. The rotation mechanism 13 is connected to a power supply, not shown, and when the practitioner turns on the power (to start the rotation operation), the rotation mechanism 13 is driven such that the body part 3 is rotated by electric power. In this way, the target tissue etc. can be effectively resected.

A preferred example of the use of the suction tube 1 is a tissue collection device comprising the suction tube 1, and a solution tank that is connected to the body part 3 and contains a sucked object. The solution tank may be connected to a hose or the like connected to the connecting part 7. For example, liquid and solid are centrifuged because of their different weights; thus, the liquid is sucked into the suction device 5, and the solid is mainly collected in the solution tank. Since the solution tank contains, for example, a tissue preservation solution, the collected tissue etc. can be analyzed.

### INDUSTRIAL APPLICABILITY

The present invention can be used in the field of medical devices.

### REFERENCE SIGNS LIST

1. Medical suction tube

3. Body part

5. Suction device

7. Connecting part

9. Blade

11. Central axis of body part

13. Rotation mechanism

1. A medical suction tube (1) having a tubular body part (3) and a connecting part (7) that connects the body part (3) to a suction device (5),
a blade (9) for resecting tissue being provided inside a tip region of the body part (3), wherein
the blade (9) is a hollow blade that is present inside the body part at an angle with respect to a central axis (11) of the body part, the hollow blade has an edge in contact with the inside of the body part, and the hollow blade is fixed to the inside of the body part,
the body part has a tip with a shape that is cut diagonally so as to be inclined with respect to the central axis of the body part,
the blade (9) is inclined with respect to the central axis of the body part along the tip of the body part with a diagonally cut shape, and
the connecting part (7) or the body part (3) has a hole (23), a suction state is continued when the hole (23) is closed, and a suction force is weakened when the hole (23) is opened.

2. The medical suction tube (1) according to claim 1, further comprising
a rotation mechanism (13) for rotating the body part (3).

3. A tissue collection device comprising:
the medical suction tube (1) according to claim 1 or 2; and
a solution tank that is connected to the body part and contains a sucked object.
